(19) United States
(12) Patent Application Publication
(10) Pub. No.: US 2022/0257166 A1
(43) Pub. Date: Aug. 18, 2022

(54) PHYSIOLOGICAL MONITORING

(71) Applicant: Siametric Systems Limited, Cambridge (GB)

(72) Inventors: David FROST, Essex (GB); Neil BAILEY, Cambridge (GB)

(21) Appl. No.: 17/628,347

(22) PCT Filed: Jul. 17, 2020
(86) PCT No.: PCT/GB2020/051722

§ 371 Date: Jan. 19, 2022

(30) Foreign Application Priority Data
Jul. 19, 2019 (GB) 1910398.5

(51) Int. Cl. A61B 5/256; A61B 5/27; A61B 5/00; A01K 13/00; B68C 1/20

(52) U.S. Cl. CPC A61B 5/256; A61B 5/27; A61B 5/6804; A61B 2562/166; B68C 1/20; A61B 2503/40; A61B 2562/225; A01K 13/006

(57) ABSTRACT

A garment or a sleeve for being worn by an animal, the garment or sleeve comprising: a structure of a flexible material; a plurality of electrical connectors for mechanically and electrically coupling to a sensing device, the connectors being attached to the structure; and a plurality of electrodes printed on a surface of the structure, each electrode being in electrical communication with a respective one of the connectors.

This invention relates to monitoring the state of an animal such as an equine, or a human.

When an animal is being trained for physical performance, or being monitored for fitness, wellbeing, health or veterinary reasons, it can be advantageous to monitor physiological functions of the animal. Examples of relevant physiological functions include heart rate, respiration rate, body temperature and a range of movement-related parameters such as speed, stride rate and acceleration in multiple axes. It may also be useful to monitor aspects of the environment in which the animal is located. Examples of relevant environmental aspects include air temperature, atmospheric particulate matter (“PM”) concentration and air oxygen concentration. It may also be of use to monitor the animal's location.

One of the problems in implementing systems for monitoring animals is establishing how to optimally mount monitoring equipment to an animal in order to achieve reliable measurements. There is a limited range of locations on an animal to which sensors can be usefully attached, and a limited range of mechanisms by which sensors can be attached. Animals can move in ways that disrupt attached equipment, for example by rolling on the ground or brushing against paddock fencing, stable walls, doors and vegetation. Riders can also accidentally knock the sensors during exercise if the sensors are not located appropriately. Furthermore, some locations at which a sensor could be attached to an animal suffer from other problems such as poor pickup of physiological signals, poor antenna performance due to attenuation by the body of the animal or exposure to the weather.

With specific reference to horses, WO 2018/002705, US 2006173367, US 20140364980 and WO 2017/216783 disclose belts, rugs, sleeves and other animal garments that can carry sensors.

There is a need for an improved medium whereby physiological and/or environmental sensing may be performed on an animal.

According to one aspect there is provided a garment or a sleeve for being worn by an animal, the garment or sleeve comprising: a structure of a flexible material; a plurality of electrical connectors for mechanically and electrically coupling to a sensing device, the connectors being attached to the structure; and a plurality of electrodes deposited on a surface of the structure, each electrode being in electrical communication with a respective one of the connectors.

The structure may be in the form of a tube. The electrodes may be on the exterior surface of the tube or sleeve.

The electrodes may be flexible.

The material of each electrode may have a greater mechanical attachment to the structure than to adjoining material of the electrode.

Each electrode may extend around and along the sleeve. It may do this in an elongate manner. It may extend helically around and along the sleeve.

Each connector may be rigid. It may, for example, be a press stud having a mushroom head, or a sprung socket for receiving such a stud.

Each connector may provide a connection interface adapted for coupling to a sensing device, the connection interface being exposed to the exterior of the sleeve. It may be exposed to the exterior of the sleeve by being outwardly directed on an exterior face of the sleeve.

The garment or sleeve may comprise an encapsulant layer overlying the electrodes and a region of the structure at least extending around the periphery of each electrode.

The electrodes may be printed on the surface of the structure.

The electrodes may be transferred on to the surface of the structure.

According to a second aspect there is provided a method for forming a garment or a sleeve for being worn by an animal, the method comprising: providing a structure of a flexible material; depositing a plurality of electrodes on to a surface of the structure; attaching to the structure a plurality of electrical connectors for mechanically and electrically coupling to a sensing device, each connector being attached to the structure such that it is in electrical communication with a respective one of the electrodes.

The electrodes may be deposited by printing a liquid material on the structure.

The liquid may comprise a conductive component and a binder.

The electrodes may be deposited by transferring a pre-printed electrode arrangement to the structure.

According to a third aspect there is provided a sleeve for being worn by an animal, the sleeve comprising: a structure of a flexible sheet material that is tubular or is configured for attaching to itself to form a tube; a plurality of electrical connectors for coupling to a sensing device, the connectors being attached to the structure; and a plurality of electrodes disposed on a surface of the structure, each electrode being electrically coupled to a respective connector and each electrode being located so that when the structure is in the form of a tube the principal direction of extension of the electrode is directed around and along the tube

The structure may be in the form of a tube. Each electrode may extend helically along the tube.

At least part of each electrode may be printed on to the structure.

The electrodes may be on the exterior surface of the sleeve.

According to a fourth aspect there is provided a garment or a sleeve for being worn by an animal, the garment or sleeve comprising: a structure of a flexible sheet material; a plurality of electrical connectors for coupling to a sensing device, the connectors being attached to the structure; and two electrodes disposed on a surface of the structure, each electrode being electrically coupled to a respective one of the connectors; each electrode being configured so that when the structure is flat an extended segment of that electrode extends linearly across the structure at an angle of between 30 and 60 degrees to an axis extending between the connectors.

The length of each extended segment may be at least 5 cm.

Each connector may be a metallic terminal configured for the snap fitting of a sensing device thereto.

Each connector may be rigid.

Each connector may extend out of the structure.

Each electrode may be flexible.

Each electrode may comprise a conductive powder adhered to the surface of the structure.

The sleeve may be a girth sleeve.

The connectors may be snap-fit plugs or sockets.

The electrodes may be adhered to the garment or sleeve by an adhesive that is resistant to softening at 80° C.

According to a fifth aspect there is provided an equine or cameline girth sleeve comprising: a tubular structure of flexible material; a plurality of snap-fit electrical connectors mounted to the structure whereby a sensor device can be attached to the sleeve; and a plurality of electrodes defined by material adhered to the exterior surface of the structure, each electrode being electrically connected to a respective one of the connectors, and each electrode extending helically around at least 25% of the circumference of the sleeve.

The structure may comprise a rubber foam material.

The electrodes may extend substantially in parallel around the sleeve.

According to a sixth aspect there is provided a handwear garment comprising: flexible sheet material defining a glove; a plurality of conductive regions exposed on the exterior of the glove, the conductive regions defining at least two electrically independent sensing zones; and a plurality of electrical connectors exposed on the exterior of the glove, each electrical connector being electrically connected to the or each conductive region of a respective one of the sensing zones.

The sheet material is may be electrically insulating. It may be a rubber foam material.

The glove may have a thumb pocket for receiving the thumb of a wearer, one or more finger pockets for receiving fingers of a wearer and a palm region for covering the palm of a wearer when the wearer's thumb is located in the thumb pocket and the wearer's fingers are located in the finger pocket(s).

The conductive regions may be located on the palm region. The conductive regions may be deposited on the surface of the palm region and/or embedded in the material of the palm region. The conductive regions may be deposited by printing. The conductive regions may be flexible.

The palm region may be on a front of the garment. The electrical connectors may be on a rear of the garment. There may be interconnects running between the two.

The glove may comprise a wrist region for encircling the wrist of a wearer when the wearer's thumb is located in the thumb pocket and the wearer's fingers are located in the finger pocket(s). The electrical connectors may be located on the wrist portion. The wrist portion may be stretchy. The connectors may be snap-fit plugs or sockets.

The glove may be configured so that the palm region covers the left palm of a wearer when the wearer's left thumb is located in the thumb pocket and the fingers of the wearer's left hand are located in the finger pocket(s). The glove may be a left glove, i.e. adapted for wear on the left hand of a typical user.

The glove may have individual finger pockets. Alternatively, it may be a mitten. The glove could be fingerless.

The handwear garment may further comprise a heart rate sensor coupled to the electrical connectors for sensing the heart rate of a subject against which the conductive regions are pressed.

The area of each conductive region may be greater than 1 cm². Each sensing zone may comprise multiple conductive regions.

The handwear garment may comprise electrical interconnects disposed on the exterior of the glove for electrically connecting the conductive regions of each sensing zone to each other and to the respective connector.

The electrical interconnects may be insulated at the exterior of the handwear garment.

At least some of the electrical interconnects may run in curves on the surface plane of the glove. The curves may be arcuate or jagged.

According to a seventh aspect there is provided a method of measuring the heart rate of an animal, the method comprising a user: wearing a handwear garment as set out above on their hand; and whilst the handwear garment remains worn on the user's hand, contacting the conductive regions against the animal.

The present invention will now be described by way of example with reference to the accompanying drawings. In the drawings:

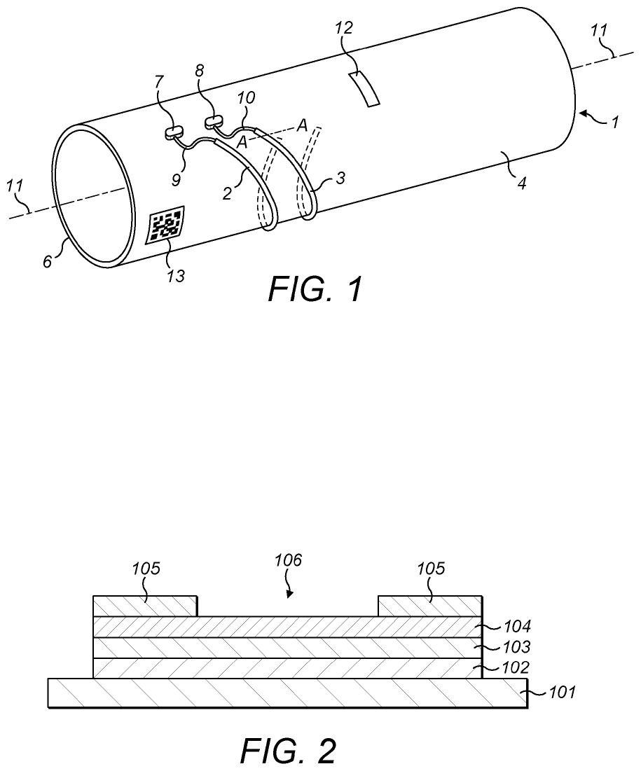

FIG. 1 shows a girth sleeve.

FIG. 2 is a cross-section of a wall of the girth sleeve of FIG. 1 on line A-A.

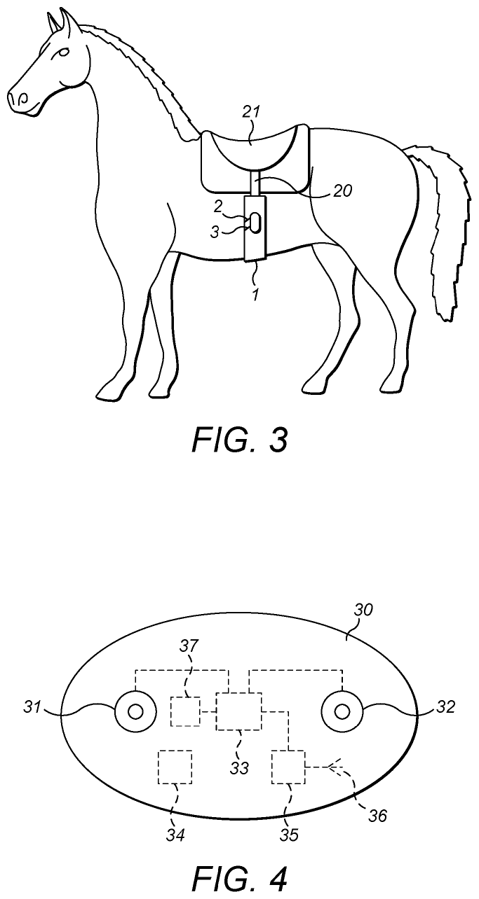

FIG. 3 shows the girth sleeve of FIG. 1 in use on a horse.

FIG. 4 illustrates a sensing device for attachment to the girth sleeve of FIG. 1.

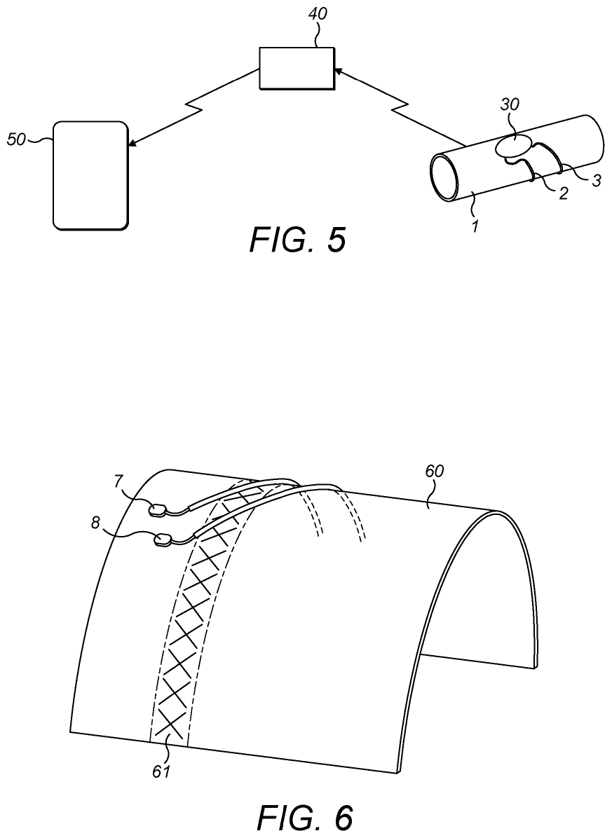

FIG. 5 shows a multi-part equine monitoring system.

FIG. 6 shows a turn-out rug adapted for equine monitoring.

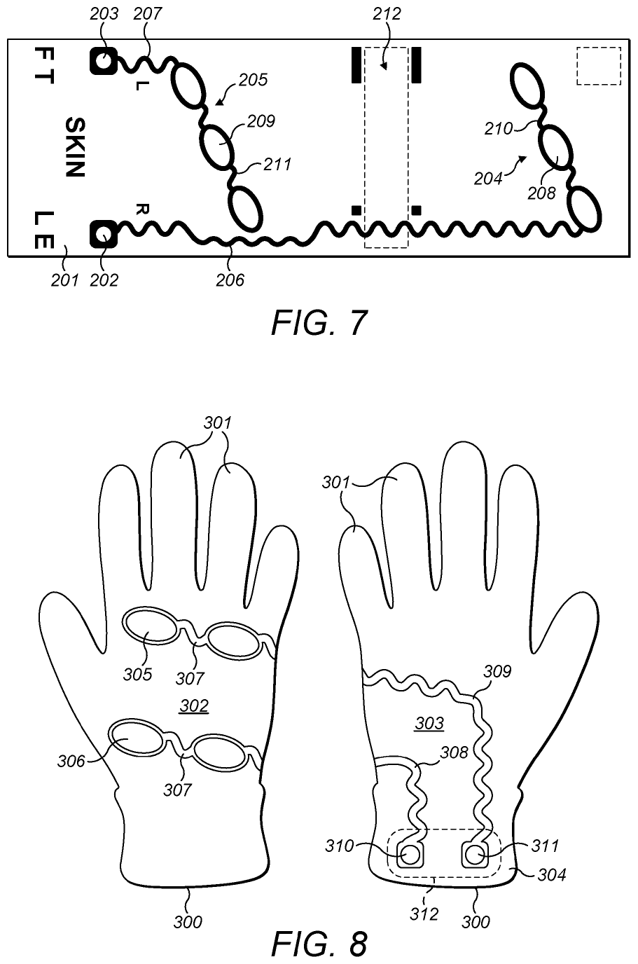

FIG. 7 shows a further embodiment of material to form a girth sleeve. The material is shown in laid-flat form. The long sides can be joined together to form a tubular sleeve.

FIG. 8 shows a glove suitable for physiological sensing.

Conventionally, a saddle is attached to a horse by means of a girth. In most cases the girth is a strap which extends from one side of the saddle to the other, running around the horse's chest. In other cases the girth is an overgirth which wraps around both the saddle and the horse's chest. The girth is tightened to hold the saddle in place. For extra security, both types of girth may be used together.

A sleeve may be applied around the girth. This can reduce the chance of the girth chafing. Additionally, when the same girth is used for multiple horses in succession, as is common in some training establishments, the girth sleeve can be changed between horses to reduce the chance of skin infections being passed from one horse to the next. Once a sleeve has been used it can straightforwardly be washed before its next use. Girth sleeves are conventionally made of towelling or neoprene material. One type of girth sleeve takes the form of a tube through which the girth is threaded. With this type of sleeve both the interior and the exterior surfaces of the girth are substantially covered by the sleeve. Another type of girth sleeve takes the form of a sheet having loops extending from one side, the girth being threaded through the loops. With this type of sleeve, the exterior of the girth is substantially exposed and the sheet is located between the interior surface of the girth and the horse's chest.

FIG. 1 shows a girth sleeve 1. The structure of the sleeve is in the form of a tube of a flexible fabric material. The material may be fabric. The material may be pliable. The material may be synthetic or non-synthetic or a composite of the two. The material may be woven or non-woven. The material may be a rubber foam. Examples of suitable materials include neoprene sheet, optionally coated with a facing fabric on one or both sides, towelling sheet or a stretch woven sheet. The tube may be formed by curving the sheet so that one edge meets the other and then joining the sheet by a technique such as stitching or welding or by using an adhesive. In this girth sleeve, electrodes 2, 3 are applied to the sleeve by printing. This approach can allow desired electrode shapes to be readily applied so the sleeve, with the electrodes being both flexible and well adhered to the sleeve.

Conveniently the length of the girth sleeve may be in the range from 500 to 1000 mm. Conveniently the circumference of the girth sleeve may be in the range from 150 to 300 mm.

A pair of electrodes 2, 3 are provided on the exterior surface 4 of the sleeve. The electrodes are insulated from each other by the structure material of the sleeve and/or by one or more encapsulant layer(s) which may be deposited on the sleeve, e.g. by printing. The electrodes are regions of conductive material that are exposed at the surface of the sleeve, allowing them to make electrical contact with objects that come into contact with the sleeve. Each electrode may comprise a single discrete region of exposed conductive material. Alternatively, each electrode may comprise multiple discrete regions of exposed conductive material, those regions being electrically interconnected by conductive connectors that are not exposed at the surface of the sleeve. Each electrode is electrically insulated from the or each other electrode. The electrodes are intended to make contact with the body of a horse that is wearing the girth sleeve in order to allow physiological measurements of the horse to be made. The electrodes may take any suitable shape. Conveniently, as shown in FIG. 1, the electrodes may be in the form of elongate strips that extend at least partially around the circumference of the girth sleeve. The electrodes may be applied to the structure of the girth sleeve by any suitable means. For example, they could be formed of conductive ink that is printed on the exterior surface of the girth sleeve. Alternatively they could be formed of conductive thread that is threaded or woven into the structure of the girth sleeve, or of conductive ribbon that is stitched or adhesively bonded to the exterior surface of the girth sleeve. Conveniently the electrodes are flexible so as to move with the sleeve when it is flexed. The electrodes may comprise a conductive powder or ink, e.g. comprising a conductor such as carbon or silver. This can enable the electrodes to flex freely with the structure of the girth sleeve whilst also being conductive.

Each electrode is electrically coupled to a connector unit 7, 8. Each connector unit may be a male or female coupler. Conveniently the connector units are rigid so as to facilitate press- or slide-fitting to another component. The connector units may be sprung to retain them to another component to which they are mated. Alternatively they may comprise an indentation into which a spring on a component to which they are mated can clip. In one convenient example the connector units are metallic studs which project outwardly from the structure material of the sleeve. The connector units may be rigid. They may extend through the structure 6 of the sleeve and be secured to it on either side.

The electrodes can advantageously be deposited on the structure of the sleeve (or on the structure of another garment for an animal) by printing or by a transfer process. In the case of printing, the electrodes can be deposited by coating the structure with a conductive ink. The ink may comprise a conductive material (e.g. in particle form) and a binder (e.g. an adhesive). The ink may be liquid when it is deposited. The conductive ink can be deposited by any suitable process, for example ink-jet printing, transfer from a print roller or gravure printing. One or more encapsulant layers may be provided on either side of the conductive ink. The or each encapsulant may be deposited form a liquid by any suitable method, e.g. ink-jet printing, transfer from a print roller or gravure printing. The encapsulant may be non-conductive.

When the electrode has been deposited by printing, typical parts of the electrode may adhere better to the structure of the sleeve or other garment than to adjoining parts of the electrode. This behaviour contrasts with the typical behaviour when an electrode is provided on a backing layer, as that backing layer will typically hold adjacent parts of the electrode together. When the electrode has been deposited by printing, the electrode may fully conform to the underlying surface of the structure of the sleeve or other garment, or to any layer located between that structure and the electrode layer. This can improve adhesion between the electrode and the structure. Printing the electrodes may provide other advantages. For example, the printed electrodes may conform well to bending and stretching of the sleeve or other garment and may readily be defined in a desired shape. A printing process may be operated at lower cost than other methods of defining electrodes.

Instead of being printed on to the structure from a liquid, the electrode may be applied as a transfer. In this process a flexible film sheet is provided. It may be a sheet of paper or film. The electrode is deposited on the film sheet. The electrode could be protected by one or more encapsulant layers on either side of it. This defines an electrode structure on the film. Adhesive is provided on the exposed face of electrode structure. The electrode structure is pressed against the article to which it is to be applied, with the adhesive layer contacting the article. Heat and/or pressure is used to activate the adhesive. The shape of the region over which the electrode is pressed on to the structure may define the resulting shape of the electrode on the structure. After the pressing process, the film sheet can be peeled away, leaving the electrode structure adhered to the article

In one embodiment the electrode may be provided in a multi-layer conductive transfer of the type described in GB 2 555 592 A. That is to say, it comprises an adhesive layer by which it is adhered to an article such as the sleeve. Disposed on that layer, in order are a first electrically insulating layer, e.g. of electrically insulating ink; an electrically conductive layer, e.g. of electrically conductive ink; and a second electrically insulating layer, e.g. of electrically insulating ink. The insulating layers act as encapsulating layers. The conductive layer is sandwiched between the insulating layers. The second, outer insulating layer may be absent in some locations to expose the conductive layer and allow connections to be made to it by contact. The structure may be provided on a backing layer or substrate (e.g. of paper or film), from which it can be transferred to an article to which it is to be attached by means of heat and/or pressure. Such a structure is shown in FIG. 2, which represents a part of the sleeve shown in FIG. 1. In FIG. 2 an article 101 (which corresponds to the body 4 of the sleeve of FIG. 1) has a multi-layer conductive structure adhered to its surface by an adhesive layer 102. The conductive structure comprises a lower insulating or encapsulant layer 103, a conductive layer 104 and an upper insulating or encapsulant layer 105. A void 106 may be defined in the outer encapsulant layer so that contact may be readily be made to the conductive layer 104.

An electrically conductive element applied to a sleeve or other garment may provide electrical interconnectivity between two connectors, or between a connector and a sensor. Alternatively or in addition it may perform an active function. In one example, the conductive element may provide substantial electrical resistance and may generate heat when current is passed through it. A garment provided with such an element may heat an animal wearing the garment. In another example, the conductive element may be such that its conductivity varies in dependence on the state of the garment. For example, its conductivity could be temperature dependent. Such an element may act as a sensor, e.g. a temperature sensor.

Ink layers or other materials of the electrode structure may be coloured or patterned. This can help to improve the visual appearance of the structure or to provide information for a user, e.g. to help in orientating the article correctly.

Connectors can be deployed on the inside and/or outside of the sleeve to facilitate convenient sensor placement. Preferred sensor locations may depend on the equine discipline that is being targeted. Positioning a sensor on the outward-facing surface of a girth sleeve or other garment can allow the sensor to be fitted more easily, e.g. whilst the garment is being worn, but may be less preferred for some disciplines (e.g. showjumping or jumps racing) due to the possibility of it snagging or being knocked by an external obstruction. In those cases, having the sensor on an inward-facing surface of the sleeve or garment may be preferred.

Multiple printed conductive elements can be applied to each of the interior and the exterior surfaces of a sleeve or other garment. Such elements on one major surface may be electrically connected to such elements on the other major surface by couplings that pass through the structure of the sleeve or other garment. These couplings may, for example, be studs.

Each connector unit may be in direct contact with a respective one of the electrodes. Alternatively, an electrically conductive lead line 9, 10 may extend between each electrode and the respective connector unit. The lead lines may be strips of conductive material that are narrower than the electrodes. These allow the electrodes and the connector units to be independently spaced apart by desired distances.

Each electrode extends around at least 25% or at least 50% or at least 60% of the circumference of the sleeve. This allows electrode material to be provided across a substantial part or the entirety of the side of the sleeve that is in contact with a horse when the sleeve is tightened to the horse's chest. Conveniently each electrode extends around from 50 to 70% of the circumference of the sleeve. This allows some freedom in positioning the sleeve on a horse.

Each electrode extends helically around the sleeve. The longitudinal axis of the strip form of each electrode has a component directed along the longitudinal axis 11 of the girth sleeve. Each electrode runs at least partially around the circumference of the girth sleeve and at least partially along the girth sleeve. By having the electrodes extend in this way, the reliability with which the electrodes can pick up physiological information is improved because they can contact a wider range of regions of the horse's body irrespective of precisely where the sleeve is positioned on a horse's chest.

Conveniently, the electrodes extend circumferentially around the sleeve (i.e. in a direction about the longitudinal axis of the sleeve) so that at least part of each electrode is offset circumferentially from all parts of the housing of the sensor. It is especially convenient if each electrode extends circumferentially around the sleeve such that at least part of it is located more than 5 cm, more than 10 cm or more than 15 cm around the circumference of the sleeve away from the housing of the sensor. This can assist in positioning the sleeve effectively on a horse because the electrodes can conveniently be located against the horse's skin whilst the sensor is not trapped between the girth, which runs through the sleeve, and the horse's skin.

Put another way, the girth sleeve has electrical connectors 7, 8 whereby a sensor device may be detachably electrically coupled to the girth. The connectors may be snap-fit connectors such as mushroom studs or sockets therefor. The connectors are of a conductive material. They may comprise a conductive metal. The connectors may be rigid. The connectors may extend out of the structure material of the girth sleeve by, for example more than 0.5 mm or more than 1 mm. A linear zone may be defined on the exterior surface of the girth sleeve. The linear zone extends fully across the girth sleeve: from one edge of the girth sleeve to another. The connectors are located outside the linear zone. Flexible electrodes extend from each connector into the linear zone. Preferably the electrodes extend at least 2 cm, at least 5 cm or at least 10 cm into the linear zone. The electrodes are disposed on the exterior surface of the girth sleeve. Animal garments can be secured to a wearing animal by a structure such as a belt or strap that encircles a part of the animal. The structure can be tightened to hold the garment to the skin of the animal. This is a widely used technique for securing animal garments. In the case of a girth sleeve the securing structure is a girth and a saddle, or an overgirth. With the arrangement of connectors and electrodes as described above, the securing structure can be tightened over the linear zone of the garment to hold the garment to an animal. This will hold the electrodes against the skin of the animal whilst leaving the connectors free. This avoids the connectors or a sensor attached over them from being pressed against the animal, which could lead to discomfort or injury. It is advantageous for at least part of each electrode to extend across the linear zone (i.e. to have an extent perpendicular to the longitudinal axis of the linear zone) and to extend along the linear zone (i.e. to have an extent parallel to the longitudinal axis of the linear zone). This can improve the effectiveness of the electrodes for picking up signals from the animal. The electrodes may, for example, comprise linear segments that extend at between 30 and 60 degrees to the longitudinal axis of the linear zone. The length of each linear segment may be at least 5 cm or at least 10 cm or at least 20 cm. The pattern in which the electrodes are disposed is considered for the relevant part of the structure of the garment being laid flat.

The electrodes may extend in parallel, or they may be non-parallel. The maximum spacing between the electrodes may be in the range from 600 to 100 mm. The minimum spacing between the electrodes may be in the range from 600 to 100 mm.

Each electrode is constituted by a region of material exposed on a surface of the sleeve or other garment, the exposed portions of that material being electrically continuous with each other. Each electrode may be in any convenient shape. Preferably it is of an elongate shape. Each electrode may take the form of a linear strip, a generally sinusoidal strip or a series of relatively wide regions (e.g. generally circular or elliptical regions) which are joined together by relatively narrow conductive necks. The relatively large regions may be arranged so as to be extended along an axis. Preferably that axis extends around and along a girth sleeve.

Conveniently the electrodes can be printed on the structure of the garment. In one process, a first layer of adhesive is deposited on the structure. Then, optionally, a lower encapsulant layer is deposited over the adhesive. This can help to resist the passage of moisture from the garment structure into the upper layers. Then a conductive layer is deposited over the adhesive and (if present) the lower encapsulant layer. Then an upper encapsulant layer is deposited over the conductive layer. Each layer may be pressed, sprayed or applied from transfer sheets on to the structure. One or other encapsulant layer may be masked to define a window through which connectors can be attached to the conductive layer. Each layer may comprise a binder whereby it can adhere to adjacent layers. The binder may initially be tacky, and may then stabilise through the release of solvent or cure through polymerisation. Each layer may be deposited in liquid form on to the structure and/or underlying layers. This can enable the layers to be formed conveniently in any suitable shape, and to be well adhered to the structure without a risk of large-scale peeling that may occur when a layer is first formed in a sheet and that sheet is then attached to the structure.

A mark 12 indicates the longitudinal centre of the girth sleeve. This can assist positioning the sleeve on a horse.

A label 13 bears machine-readable indicia that uniquely identify the sleeve. The indicia may be a QR code, a one-dimensional or a two-dimensional bar code. Alternatively, the label may comprise a radio frequency identification tag that uniquely identifies the sleeve. This can assist in associating the sleeve with a horse, as will be described below.

In a preferred embodiment, the structure material of the sleeve is neoprene, coated with a facing fabric on at least the face that is directed to the exterior of the sleeve. The electrodes are strips of a width between 15 and 30 mm, and extending around 50 to 60% of the circumference of the sleeve. The strips are angled at between 40 and 70% to the longitudinal axis of the sleeve. The strips are parallel and spaced apart by around 100 to 200 mm. The connector units are female metallic sockets which face outwardly from the outer face of the structure of the sleeve.

Instead of neoprene, other materials may be used. Examples include woven or knitted fabrics (e.g. of cotton, nylon or elastic polymers such as Lycra) leather or rubber.

The connector units can be connected to an electronic sensor for sensing physiological data that is picked up by the electrodes. In one example, the sensor may be a heart rate monitor. The connector units may be 5 mm sockets (or another size). They may be spaced apart by approximately 50 mm. This can allow a commercially available heart rate monitor sensor such as a Wahoo Tickr, Polar H7/H9/H10 or a Garmin HRM-Run or a proprietary heart rate monitor to be attached to the sleeve. The sensor can store sensed data locally for subsequent download to another device for analysis. Alternatively it may wirelessly transmit an indication of sensed data in real time, e.g. using a short-range wireless protocol such as Bluetooth, Bluetooth LE or ANT+.

The sleeve may be used in the following way. A girth 20 (see FIG. 3) is attached at one of its ends to one side of a saddle 21. The sleeve 1 is threaded on to the girth. The saddle is placed on a horse and the free end of the girth is attached to the other side of the saddle. The girth is tightened around the horse's chest so as to secure the saddle on the horse's back, with the sleeve positioned such that:

1. it is substantially central laterally with respect to the horse: this can be achieved with the aid of the central mark 12;

2. the connector units are facing away from the horse's body: this facilitates the attachment of a sensor unit to the sleeve after the girth has been tightened and avoids the sensor being pressed against the horse and/or trapped between the girth and the horse's chest; and

3. at least part of each electrode is facing the horse's body and preferably is trapped between the girth and the horse's chest since then it can be held firmly in contact with the horse's chest by the girth. It will be noted that because the electrodes extend circumferentially around the sleeve in such a way as to be circumferentially offset from the connector units, the electrodes can be in contact with the body of the horse even though the connector units are facing away from the horse's body.

Then a suitable sensor can be clipped to the connector units. Alternatively the sensor can be attached to the sleeve at an earlier stage. Now the horse can be exercised, turned out or left in a stable, and the sensor can capture physiological information about the horse by means of the electrodes.

The sensor is detachably secured to the sleeve when in use. This means that the sensor can be detached when the sleeve is washed, or transferred from one sleeve to another.

FIG. 4 shows a suitable sensor. In this example the sensor is a heart rate monitor. The sensor comprises a housing 30. Connectors 31, 32 are exposed at the exterior of the housing. Those connectors are configured for releasable connection to the connectors 7, 8 of the sleeve. Inside the housing are a processor 33, a battery 34, a transmitter 35 and an antenna 36. The processor is electrically coupled to the connectors 31, 32 and is arranged to sense heart rate information in dependence on electrical signals picked up by the electrodes of the sleeve and conveyed via the connectors 31, 32. The processor forms data representing the sensed heart rate information and passes it to the transmitter for transmission via the antenna 36. The data can then be received remotely for analysis. Alternatively, the data can be cached in a memory 37.

The sensor may sense any appropriate parameter or parameters. It may sense a physiological parameter of an animal wearing the sensor. The parameter may be sensed by means of the electrodes. Examples of parameters that can be sensed include physiological parameters such as heart rate, respiration rate, body temperature, movement-related parameters such as speed, stride rate and acceleration in multiple axes and environmental parameters such as air temperature, atmospheric particulate matter (“PM”) concentration and air oxygen concentration. It may also monitor the animal's location.

The sensor may continuously or periodically transmit the sensed data or a representation of it, which may be simplified to save bandwidth. The data may be transmitted by any suitable mechanism, for example IEEE 802.11 or Bluetooth. Alternatively the sensor may cache the sensed data or a representation of it in a memory local to the sensor. That data may later be downloaded over a wired or wireless connection for analysis. In one convenient arrangement, the sensor transmits the sensed data or a representation of it over a relatively short-range protocol (e.g. IEEE 802.11, Bluetooth or Bluetooth LE) as the data is sensed. The horse carries a relay transceiver 40 (see FIG. 5) which is capable of receiving data transmitted over the relatively short-range protocol and transmitting it over a relatively long range protocol (e.g. a cellular telephony protocol). This allows the hardware 40 for the relatively long range protocol to be carried separately from the sensor and the garment to which it is mounted, which may make the garment easier to position and may improve the range of transmission of signals in the relatively long range protocol. Conveniently the relay transceiver may be mounted on the horse's head, poll or neck, for example by being attached to a headcollar or bridle. The relay transmitter transmits the data to a remote unit 50 such as the cloud or a cellular telephone for notifications, visualisation and analysis.

In the examples above, the sensor is attached to a girth sleeve. The sensor may be attached to sleeves or garments or other tack suitable for being worn by equines or other animals. Non-limiting examples include rugs (e.g. stable rugs, turnout rugs, fly sheets and cooling rugs), saddles, collars, headcollars, bridles, under-saddle garments (e.g. saddle cloths, numnahs and saddle pads), surcingle, saddles, full blankets, half blankets, stable blankets, night blankets, vests, rain sheets, coolers, anti-sweat sheets, therapeutic blankets, under rugs, half-sheets, quarter sheets, rump rugs, blinkers, nose pads, poll pads, protective boots and reins, The garment could be for any suitable animal. The garment could be for a quadruped such as an equine or a canine. FIG. 6 shows the example of an equine rug 60. The rug has connectors 7, 8 for a sensor and electrodes 2, 3 which extend diagonally or helically across the rug from the connectors. A linear zone 61 extends across the rug from one edge to the opposite edge. The connectors 7, 8 lie outside that zone, but both electrodes include portions that lie inside that zone. This allows the rug to be tightened to a horse by a strap running around that zone which can hold the electrodes to the skin of the horse without pinching the connectors or a sensor attached to the connectors against the horse. The side of the rug shown as upward-facing in FIG. 6 would be placed against the horse when the rug is in use.

The fact that the electrodes extend helically can help in providing good contact to the horse since each electrode is exposed to a range of locations on the animal that are subject to different conditions. Those locations vary along the length of the horse and circumferentially around the body of the horse. This can help in avoiding disruption of electrical contact due to factors such as dirt or sweat, or if the sleeve or other garment on which the electrodes are applied twists either about its own axis or relative to the horse.

Incorporating the electrodes onto the material of a sleeve or other garment means that unlike free cables they are unlikely to become snagged on obstructions. It can also reduce the likelihood of chafing on the animal.

It is convenient for the article to be capable of withstanding washing at conventional temperatures without substantial deterioration. The adhesive used for the electrode structure, and any protective layers of the electrode structure, may be selected so that they are stable up to a temperature of, e.g. at least 80° C.

FIG. 7 shows an example layout for a girth sleeve. FIG. 7 shows the material 201 to form the sleeve laid flat. To form the sleeve the material would be bent into a tube and the long edges of the material would be joined together. A set of conductive structures are disposed on the surface of the material that is to form the outer surface of the sleeve when the sleeve is in use. Those structures comprise conductive connector points 202, 203 for connection to a sensor device such as a heart rate sensor. The connector points are conveniently disposed at similar locations along the length of the sleeve, and preferably within 25% of the length of the sleeve from one end. That can enable a sensor to be coupled to the connectors without the sensor being exposed at the lower part of the horse's chest when the sleeve is in use. This reduces the risk of the connector hitting on a jump that the horse might negotiate. Leads 206, 207 extend from respective connector points to respective electrodes which are indicated generally as 204, 205. Each lead is formed of a trace of conductive material. The traces are preferably thinner than the connector points since that can avoid the use of unnecessary conductive material for the traces whilst permitting the connector points to provide some flexibility in coupling locations. Each electrode comprises a series of relatively enlarged conductive regions 208, 209 which are interconnected by relatively narrow interconnects 210, 211. All the conductive regions of each electrode are electrically coupled together in series by interconnects, and to the respective connector point by the respective lead 206, 207. The relatively enlarged regions have an average width that is greater than that of the interconnects. In each relatively enlarged region conductive material is exposed to the exterior, for example as illustrated at 106 in FIG. 2. The relatively enlarged regions of each electrode extend in a line in a direction that is angled to the longitudinal axis of the material 101. This results in the electrodes extending helically around the sleeve when the material is formed into a tube. The electrodes may run helically in the sense that each electrode lies within a rectangular zone on a surface (preferably the exterior surface) of the sleeve, the length of the zone being at least three or five times its width, and the electrode intersecting the longitudinal ends of that zone. Each electrode may lie exclusively in such a zone. Indicia such as those shown at 212 can assist in the identification and/or positioning of the sleeve.

The tracks 206, 211 etc. are of a sinuous form. Each track incorporates one or more curves. The length of each track is greater than the straight-line length between the ends of the respective track. This can assist in making the tracks more resistant to disruption if the material of the sleeve is stretched.

The structure material of the garment, or a region deposited on the outer surface of the structure material of the sleeve could be thermochromic. This can allow it to provide a visual indication of the body temperature of the animal wearing the garment.

There may be multiple sensors attached to a single sleeve or other garment. For example, both a heart rate sensor and a dust sensor may be attached simultaneously.

One or more pockets may be provided on the sleeve to help hold one or more sensors in place.

Embodiments have been discussed above in relation to garments (e.g. girth sleeves) for horses in particular. Analogous garments could be used for other animals, especially but not exclusively racing animals. Non-limiting examples include horses, equids other than horses, camels and dogs.

FIG. 8 shows an alternative form of garment. The garment of FIG. 8 is a glove configured to be worn on the hand of a human. FIG. 8 shows the front and back of the glove. The glove is a pouch open at one end 300 and formed with individual elongate pockets 301 to accommodate the digits of a human wearing the glove. For clarity only some of the pockets are labelled in FIG. 8. The front of the glove has a palm region 302 for covering the palm of a wearer's hand. The rear of the glove has a dorsal region 303 for covering the dorsal aspect of the hand. The glove has a wrist region 304 for encircling the wrist of a wearer. The wrist region may be elasticated to help hold the glove on the hand of a wearer. The glove is formed of a flexible material such as a fabric or foam rubber material. Conveniently, the material of the body of the glove is electrically insulating.

On one face of the glove, conveniently the front, are disposed electrode pads 305, 306. Each electrode pad is constituted by conductive material exposed at the exterior surface of the glove. Each electrode pad may be integrated with the material of the body of the glove (e.g. by being threaded, woven or moulded into that material) or may be adhered to the surface of the material of the body of the glove (e.g. by being printed or otherwise deposited on that material). The electrode pads may be of any suitable shape. In one example the pads may be of an oval shape. The electrode pads define two independent electrically conductive paths. Each path may comprise one or more electrode pads. In the example of FIG. 8 each path comprises two oval electrode pads. Where a path has multiple electrode pads they may be electrically interconnected by interconnects 307. The interconnects may be straight. Alternatively they may have a curved and/or sinuous shape. With a curved/sinuous shape the interconnects may be better able to flex or stretch to accommodate movement of the material of the glove. Each interconnect may be constituted by conductive material exposed at the exterior surface of the glove. Alternatively, the conductive material of the interconnect may be covered by an insulator so that it is not exposed at the exterior of the glove. This may protect the interconnects. Each interconnect may be integrated with the material of the body of the glove (e.g. by being threaded, woven or moulded into that material) or may be adhered to the surface of the material of the body of the glove (e.g. by being printed or otherwise deposited on that material). For each conductive path a further interconnect 308, 309 leads to a respective connector 310, 311. The connectors are configured to attach to a sensing device, e.g. of the type discussed above and illustrated in FIG. 4. Each connector may be of any suitable form: for example a plug, socket, press stud or press socket. The connectors may be on the rear of the glove. The connectors may be exposed or may be covered with a pocket in the region 312 where the connectors are located. The pocket may help to hold a sensing device in place when it is attached to the connectors.

The interconnects may be more flexible than the electrode pads.

In operation, a sensor is attached to the connectors in the manner described above, and a user wears the glove on their hand. The user can then press the glove against a subject (e.g. an animal or human) to sense a physiological state of the subject. As in the device of FIG. 1, the physiological state may be sensed in dependence on current flow between the electrode pads of the respective conductive paths. The physiological state may be heart beats or the frequency of them, or signals indicative of local muscle activity. The subject may be an equine or another animal e.g. of the types discussed above.

The material of the body of the glove may be a rubber such as neoprene. The conductive pads and/or interconnects may be deposited by a conductive print transfer process. Conductive transfers may be printed on a transfer film and applied to a die-cut piece of glove body material using pressure and heat. The edges of the body material can then be glued and/or stitched to form a glove.

The glove may be used to acquire an ECG or monitor heart rate (e.g. resting or recovery heart rate) of the subject. In the case of an equine subject, the subject may conveniently be stationary, without any tack, for instance in its stable, yard or horse box. Since the heart of a typical horse is on the left side of its body, the glove is conveniently a left-hand glove. That is: viewing the palm region of the glove, where the conductive pads are, with the wrist downwards, the thumb pocket is on the left. Conveniently the connectors/terminals 310, 311 are at the wrist of the glove and/or on the rear of the glove. This can make the glove more comfortable to wear.

By providing a sensing device in the form of a glove, a user can collect physiological data about the subject in a way that can avoid imposing stress on the subject. The user can calmly and easily hold their gloved hand against the subject to make a measurement. This approach is natural and may simply be part of stroking an animal. The sensor may be re-used on the device shown in FIG. 1.

The glove may have conjoined fingers as in a mitten.

The connectors may be configured as described for previous embodiments. The body material of the glove and the material(s) of the electrical elements may be as described for previous embodiments. The method by which the electrical elements are disposed on the body material of the glove may be as described for previous embodiments.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A garment or a sleeve for being worn by an animal, the garment or sleeve comprising:
a structure of a flexible material;
a plurality of electrical connectors for mechanically and electrically coupling to a sensing device, the connectors being attached to the structure; and
a plurality of electrodes deposited on a surface of the structure, each electrode being in electrical communication with a respective one of the connectors.

2. The sleeve as claimed in claim 1, wherein the structure is in a form of a tube and the electrodes are on an exterior surface of the sleeve.

3. The garment or sleeve as claimed in claim 1, wherein the electrodes are flexible.

4. The garment or sleeve as claimed in claim 1, wherein material of each electrode has a greater mechanical attachment to the structure than to adjoining material of the electrode.

5. The sleeve as claimed in claim 1, wherein each electrode extends around and along the sleeve.

6. The garment or sleeve as claimed in claim 1, wherein each connector is rigid.

7. The sleeve as claimed in claim 1, wherein each connector provides a connection interface adapted for coupling to a sensing device, the connection interface being exposed to an exterior of the sleeve.

8. The garment or sleeve as claimed in claim 1, comprising an encapsulant layer overlying the electrodes and a region of the structure at least extending around a periphery of each electrode.

9. The garment or sleeve as claimed in claim 1, the electrodes being printed on a surface of the structure.

10. The garment or sleeve as claimed in claim 1, the electrodes being transferred on a surface of the structure.

11. A method for forming a garment or a sleeve for being worn by an animal, the method comprising:
providing a structure of a flexible material;
depositing a plurality of electrodes on to a surface of the structure;
attaching to the structure a plurality of electrical connectors for mechanically and electrically coupling to a sensing device, each connector being attached to the structure such that it is in electrical communication with a respective one of the electrodes.

12. The method as claimed in claim 11, wherein the electrodes are deposited by printing a liquid material on the structure.

13. The method as claimed in claim 12, wherein the liquid comprises a conductive component and a binder.

14. The method as claimed in claim 11, wherein the electrodes are deposited by transferring a pre-printed electrode arrangement to the structure.

15. A sleeve for being worn by an animal, the sleeve comprising:
a structure of a flexible sheet material that is tubular or is configured for attaching to itself to form a tube;
a plurality of electrical connectors for coupling to a sensing device, the connectors being attached to the structure; and
a plurality of electrodes disposed on a surface of the structure, each electrode being electrically coupled to a respective connector and each electrode being located so that when the structure is in a form of a tube and a principal direction of extension of the electrode is directed around and along the tube.

16. The sleeve as claimed in claim 15, wherein when the structure is in the form of the tube and each electrode extends helically along the tube.

17. The sleeve as claimed in claim 15, wherein at least part of each electrode is printed on to the structure.

18. The sleeve as claimed in any of claim 15, wherein the electrodes are on an exterior surface of the sleeve.

19-45. (canceled)
